SEQUENCE LISTING (13 sequences)

SEQ ID NO: 1 - DNA, 31 bp, Artificial Sequence
  Primer dimer sequence
atcctcctgggccatctcctggaaacctgtt

SEQ ID NO: 2 - DNA, 38 bp, Artificial Sequence
  PCR primer (HRR_0628_F)
aagactcggcagcatctccaatcctcctgggccatctc

SEQ ID NO: 3 - DNA, 40 bp, Artificial Sequence
  PCR primer (HRR_0764_R)
gcgatcgtcactgttctccaaacaggtttccaggagatgg

SEQ ID NO: 4 - DNA, 38 bp, Artificial Sequence; identical to SEQ ID NO: 2
  PCR primer (HRR_0628_F)

SEQ ID NO: 5 - DNA, 40 bp, Artificial Sequence; identical to SEQ ID NO: 3
  PCR primer (HRR_0764_R)

SEQ ID NO: 6 - DNA, 38 bp, Artificial Sequence; identical to SEQ ID NO: 2
  PCR primer (HRR_0628_F)
  misc_feature (30)..(31): RNA bases

SEQ ID NO: 7 - DNA, 40 bp, Artificial Sequence; identical to SEQ ID NO: 3
  PCR primer (HRR_0764_R)
  misc_feature (32)..(33): RNA bases

SEQ ID NO: 8 - DNA, 42 bp, Artificial Sequence
  PCR primer (ADHv3_0265-F)
aagactcggcagcatctccatgtttaccatttgttggcagag

SEQ ID NO: 9 - DNA, 37 bp, Artificial Sequence
  PCR primer (ADHv3_0323-R)
gcgatcgtcactgttctccaccccgcctctgcccaac

SEQ ID NO: 10 - DNA, 42 bp, Artificial Sequence; identical to SEQ ID NO: 8
  PCR primer (ADHv3_0265-F)

SEQ ID NO: 11 - DNA, 37 bp, Artificial Sequence
  PCR primer (ADHv3_0245-F)
gcgatcgtcactgttctccacccatcgctccgtctct

SEQ ID NO: 12 - DNA, 42 bp, Artificial Sequence
  PCR primer - with RNA base substitutions
  misc_feature (31)..(32): RNA bases
aagactcggcagcatctccatgtttaccatuugttggcagag

SEQ ID NO: 13 - DNA, 42 bp, Artificial Sequence; identical to SEQ ID NO: 8
  PCR primer - with RNA base substitutions
  misc_feature (37)..(38): RNA bases

(19) United States
(12) Patent Application Publication
(10) Pub. No.: US 2023/0212559 A1
(43) Pub. Date: Jul. 6, 2023

(54) CHIMERIC PRIMERS AND RELATED METHODS

(71) Applicant: Agilent Technologies, Inc., Santa Clara, CA (US)

(72) Inventors: Jurgen DEL FAVERO, Niel (BE); Lien HEYRMAN, Niel (BE); Dirk GOOSSENS, Niel (BE); Bart TEGENBOS, Niel (BE)

(21) Appl. No.: 17/790,094

(22) PCT Filed: Dec. 30, 2020
(86) PCT No.: PCT/EP2020/088041

§ 371 Date: Jun. 29, 2022

Related U.S. Application Data
(60) Provisional application No. 62/955,253, filed on Dec. 30, 2019.
(60) Provisional application No. 62/955,260, filed on Dec. 30, 2019.

(51) Int. Cl. C12N 15/10; C12Q 1/6853; C12Q 1/686

(52) U.S. Cl. CPC C12N 15/1089; C12Q 1/686; C12Q 1/6853

(57) ABSTRACT

The present disclosure provides chimeric primers suitable for use in the amplification of a nucleic acid sequence. In some aspects, these chimeric primers reduce the formation of primer dimers and/or off-target amplification products, compared to amplification reactions carried out using unmodified primers.

### CROSS-REFERENCE TO RELATED APPLICATIONS

This application claims the benefit of U.S. Provisional Application Serial Nos. 62/955,253 and 62/955,260, which were filed on Dec. 30, 2019, and the entire contents of each of which is expressly incorporated by reference herein.

### TECHNICAL FIELD

The disclosure generally relates to compositions, methods, and kits for reducing non-specific nucleic acid amplification for use in the field of nucleic acid amplification and detection.

### BACKGROUND

Polymerase chain reaction (PCR) methods are core to a variety of diagnostic methods, e.g., high-throughput SNP genotyping, and serve as a foundation for applications in forensic analysis, including human identification and paternity testing, the diagnosis of infectious diseases, diagnosis and prognosis of diseases through NGS, and pharmacogenomic studies aimed at understanding the connection between individual genetic traits, drug response and disease susceptibility.

The efficiency of many PCR methods, particularly those that employ multiplex PCR methods in which several different products are amplified in a single reaction, is often low because primers may hybridize (or “anneal”) to each other, rather than to the template to be amplified. These off-target interactions may generate non-specific amplification products as a template-independent artifact of a PCR amplification reaction. As such, the specificity of primer-based amplification methods depends in large part on the specificity of primer hybridization and extension. Under the elevated temperatures used in a typical PCR amplification, each pair of primers will normally hybridize only to the intended target sequence. However, due to the high number of different primers in a multiplex PCR and their high availability in the reaction medium, many short-lived interactions between primers do occur and the chance exists that the DNA polymerase extends primers based on these interactions. Once these extension products (i.e., primer dimers) are formed, they will be preferentially amplified due to their smaller size.

Non-specific primer extension products compete with the amplification of the desired target sequence(s) and can significantly decrease the efficiency of the amplification of the desired sequence. One commonly observed type of non-specific amplification product is a template-independent artifact of amplification reactions often referred to as “primer dimer.” Primer dimers are double-stranded fragments formed from the hybridization and extension of pairs of primers in the PCR reaction mixture. The resulting extension product forms a template which, because of its short length, is amplified efficiently. As such, researchers interested in large multiplex PCR assays must often devote a significant amount of time and resources towards the design, testing and refinement of PCR primers and assay conditions. Unfortunately, current methods fail to offer a generally-applicable solution (e.g., rules for primer design that can be consistently applied to PCR assays to mitigate this issue), necessitating a substantial amount of trial and error until suitable assay parameters are identified.

### SUMMARY OF VARIOUS EMBODIMENTS

In a general aspect, the disclosure provides methods, compositions, and kits that may be used to amplify DNA in a DNA-dependent polymerase amplification method (e.g., using multiplex PCR), while advantageously reducing or eliminating the formation of primer dimers and/or non-specific amplification products. While the disclosure typically refers to PCR as an example, it is understood that the chimeric primers described herein (and the methods for designing such primers) can also be used with exponential rolling circle amplification (ERCA), rolling circle amplification (RCA), multiple displacement amplification (MDA), strand displacement amplification (SDA), nucleic acid sequence based amplification (NASBA), transcription-mediated amplification (TMA), real-time quantitative PCR (qPCR), self-sustained sequence replication (3SR), amplification with Qβreplicase, and cycle sequencing.

In one aspect, the disclosure relates to a method for generating chimeric primers capable of amplifying at least a portion of a template DNA molecule, while minimizing or eliminating non-specific amplification, comprising: a) identifying a non-specific amplification fragment produced during a PCR assay; b) identifying one or more DNA primers that produce the non-specific amplification fragment; and c) selecting a sequence for at least one chimeric primer; and optionally, d) generating the at least one chimeric primer; wherein the chimeric primer is an oligonucleotide comprising DNA and RNA bases, and has a sequence identical to that of one of the identified DNA primers except for a first segment wherein at least two adjacent DNA bases are replaced by corresponding RNA bases. The chimeric primer may be generated using any oligonucleotide synthesis method known in the art.

In some aspects, the first segment is located outside of an overlapping region produced when the one or more DNA primers identified in step b) hybridizes with one or more other DNA primers during the PCR assay to form a primer dimer. In some aspects, the overlapping region is determined based on hybridization conditions compatible with a PCR assay.

In some aspects, the corresponding RNA bases are located within 15 bases of the 3′ end of the sequence, and the sequence has a DNA base at its 3′ end.

In some aspects, the sequence of the chimeric primer further comprises a second segment comprising at least two adjacent DNA bases replaced by corresponding RNA bases, wherein the first segment and the second segment are separated by at least two DNA bases. The first segment and the second segment may be located within 15 bases of the 3′ end of the sequence of the selected sequence.

In some aspects, the sequence of the at least one chimeric primer further comprises a third segment comprising at least two adjacent RNA bases that replace corresponding DNA bases in the identified DNA primer, wherein the second segment and the third segment are separated by at least two DNA bases.

In some aspects, selecting a sequence for at least one chimeric primer comprises: selecting a sequence for a first chimeric primer configured to hybridize to an upstream portion of the template DNA molecule; and selecting a sequence for a second chimeric primer configured to hybridize to a downstream portion of the template DNA molecule; wherein the first and second chimeric primers are capable of amplifying at least a portion of the template DNA molecule by PCR.

In another general aspect, the disclosure provides a method for, generating chimeric primers capable of amplifying DNA while minimizing or eliminating the formation of primer dimers, comprising: a) identifying a primer dimer produced during a PCR assay; b) identifying one or more DNA primers that produce the primer dimer; c) identifying an overlapping region produced when the one or more DNA primers are hybridized; and d) selecting a sequence for at least one chimeric primer; wherein the chimeric primer is an oligonucleotide comprising DNA and RNA bases, and has a sequence identical to that of one of the identified DNA primers except for a first segment wherein at least two adjacent DNA bases are replaced by corresponding RNA bases.

In some aspects, the first segment is located outside of the overlapping region produced when the one or more DNA primers identified in step b) hybridize(s) with one or more other DNA primers during the PCR assay to form a primer dimer. In some aspects, the overlapping region is determined based on annealing conditions compatible with a PCR assay (e.g., high- or low-stringency conditions).

In some aspects, the at least one chimeric primer is configured to amplify at least a portion of the template DNA while reducing or eliminating formation of non-specific amplification products.

In some aspects, the PCR assay is a multiplex PCR assay that generates an end product comprising: a) less than 5% primer dimer amplification products; b) less than 7% primer dimer amplification products; or c) less than 10% primer dimer amplification products

In another general aspect, the disclosure provides chimeric primers generated using any of the methods described herein, as well as kits comprising the same.

In yet another general aspect, the disclosure relates to a method for amplifying DNA, comprising: a) conducting a PCR assay using a reaction mixture comprising one or more chimeric primers, a DNA-dependent polymerase, and a template DNA molecule; and b) amplifying at least a portion of the template DNA molecule using the one or more chimeric primers; wherein each of the one or more chimeric primers is an oligonucleotide comprising DNA and RNA bases, having a sequence that includes a first segment comprising at least two adjacent RNA bases.

In some aspects, at least one of the one or more chimeric primers comprises a sequence having a pair of adj acent RNA bases spanning positions 3 and 4, 7 and 8, or 14 and 15, as measured from the 3′ end of the chimeric primer. In some aspects, the remainder of the sequence consists of DNA bases.

In some aspects, the first segment is located within 15 bases of the 3′ end of the sequence, said sequence having a DNA base at its 3′ end.

In some aspects, the sequence of at least one of the chimeric primers further comprises a second segment comprising at least two adjacent RNA bases, wherein the first segment and the second segment are separated by at least two DNA bases (e.g., the first segment may span positions 3 and 4, and the second segment may span either positions 7 and 8, or 14 and 15, as measured from the 3′ end of the chimeric primer).

In some aspects, the sequence of at least one of the chimeric primers further comprises a third segment comprising at least two adjacent RNA bases, wherein the second segment and the third segment are separated by at least two DNA bases (e.g., the first segment may span positions 3 and 4, the second segment may span positions 7 and 8, and the third segment may span positions 14 and 15, as measured from the 3′ end of the chimeric primer).

In some aspects, the reaction mixture comprises two chimeric primers, and each chimeric primer is an oligonucleotide comprising DNA and RNA bases with a sequence that includes a first segment comprising at least two adjacent RNA bases.

In some aspects, the reaction mixture comprises a plurality of chimeric primers, wherein the first segment of each chimeric primer is located within 15 bases of the 3′ end of the sequence of each respective chimeric primer, each sequence having a DNA base at its 3′ end. In some aspects, the sequence of each chimeric primer further comprises a second segment comprising at least two adjacent RNA bases, wherein the first segment and the second segment are separated by at least two DNA bases. In some aspects, the sequence of each chimeric primer further comprises a third segment comprising at least two adjacent RNA bases, wherein the second segment and the third segment are separated by at least two DNA bases. In some aspects, at least one of the first segment, the second segment, and the third segment are located within 15 bases of the 3′ end of the sequence of each chimeric primer.

In still further aspects, the one or more chimeric primers in the reaction mixture comprise: a forward primer configured to hybridize to an upstream portion of the template DNA molecule; and a reverse primer configured to hybridize to a downstream portion of the template DNA molecule; wherein the forward primer and the reverse primer are chimeric oligonucleotides, each comprising DNA and RNA bases and a sequence that includes a first segment comprising at least two adjacent RNA bases.

In some aspects, the forward primer and the reverse primer each comprise a sequence having a DNA base at its 3′ end, and wherein the first segment of each sequence is located within 15 bases of its respective 3′ end.

In some aspects, at least one of the forward primer and the reverse primer further comprises a second segment comprising at least two adjacent RNA bases, wherein the first segment and the second segment are separated by at least two DNA bases.

In some aspects, both the forward primer and the reverse primer each further comprise a second segment comprising at least two adjacent RNA bases, wherein the first segment and the second segment are separated by at least two DNA bases.

In some aspects, the one or more chimeric primers are configured to amplify at least a portion of the template DNA while reducing or eliminating formation of non-specific amplification products.

In some aspects, one or more chimeric primers are configured to amplify at least a portion of the template DNA while reducing or eliminating primer dimers and/or off-target amplification.

In some aspects, the PCR assay is a multiplex PCR assay that generates an end product comprising less than 5, 6, 7, 8 , 9 or 10% primer dimer amplification products.

In some aspects, the PCR assay is a multiplex PCR assay that generates an end product comprising: a) less than 5% primer dimer amplification products; b) less than 7% primer dimer amplification products; or c) less than 10% primer dimer amplification products

In another general aspect, the disclosure provides methods for designing chimeric primers, comprising: a) selecting a DNA primer from a pair of DNA primers configured to amplify at least a portion of a template DNA molecule in a PCR assay; b) selecting a sequence for a chimeric primer, wherein the chimeric primer is an oligonucleotide comprising DNA and RNA bases, having a sequence that includes a first segment consisting of two adjacent RNA bases, said sequence being identical to the sequence of the DNA primer selected in step a) except for the first segment; and c) optionally, generating the chimeric primer (e.g., using any known oligonucleotide synthesis method). In some aspects, the first segment spans positions 3 and 4, 5 and 6, 6 and 7, 7 and 8, 8 and 9, 9 and 10, 10 and 11, 11 and 12, 12 and 13, 12 and 14, or 14 and 15, as measured from the 3’′ end of the chimeric primer. In other aspects, the first segment may span any two adjacent bases of the chimeric primer.

In another general aspect, the disclosure provides chimeric primers generated using any of the methods described herein, as well as kits comprising the same.

Additional aspects will be readily apparent to one of skill in light of the totality of the disclosure.

### BRIEF DESCRIPTION OF THE DRAWINGS

FIG. 1 is a chart summarizing the results of a series of PCR assays conducted using either DNA primers or at least one chimeric primer. Shaded boxes indicate primer pairs which resulted in primer dimer formation, where at least one primer in the pair was a chimeric primer. This figure also illustrates differences in the level of primer dimer formation in chimeric primers having at least two non-adjacent RNA bases (i.e., the “Peleg” approach, described in further detail below) versus at least two adjacent RNA bases in accordance with the present disclosure.

FIG. 2 is a chart summarizing the results of a series of PCR assays conducted using either DNA primers (unshaded boxes) or at least one chimeric primer (shaded boxes) having either one, two, or three RNA bases 5′ of the overlapping region of each respective primer pair.

FIG. 3 is a chart summarizing the results of a series of PCR assays conducted using either standard DNA primers (unshaded boxes) or at least one chimeric primer (shaded boxes), which illustrates the relative coverage of various amplicons produced using these primer pairs.

FIG. 4 shows three chromatograms analyzing the results of a PCR using either: standard universal primers on genomic DNA (top), or two different pairs of chimeric primers (middle, bottom). As shown by this figure, no non-specific fragments were detected when the chimeric primer pairs were used, indicating increased specificity.

FIG. 5 shows three chromatograms analyzing the results of a 30-cycle PCR conducted without genomic DNA in the reaction mix, using either: standard universal primers (top), or two different pairs of chimeric primers (middle, bottom). As shown by this figure, no non-specific fragments were detected when the chimeric primer pairs were used. In contrast, the standard universal primers interacted with each other (e.g., forming primer dimers), resulting in the generation of non-specific amplification products.

FIG. 6 depicts an exemplary workflow for generating chimeric primers according to the disclosure, highlighting the location of the overlapping region between this representative pair of primers.

FIG. 7 is a chart showing an exemplary set of three DNA primers that generate two potential primer dimers in a PCR assay, each having a different overlapping region.

FIG. 8 is a chart summarizing the results of a series of PCR assays conducted using a pair of primers, in their original form (“all DNA”) or modified to incorporate at least one RNA base at specific positions (rows “RNA test 1” through “RNA test 5”).

FIG. 9 is a chart summarizing the results of a series of PCR assays conducted using multiple pair of primers, in their original form (“all DNA”) or modified to incorporate a pair of RNA bases at specific positions (columns “Version 1” through “Version 3”). Shaded boxes indicate instances where a DNA primer was replaced by a chimeric primer.

FIG. 10 shows three graphs illustrating the relative amplicon coverage of the chimeric primers shown in FIG. 3.

FIG. 11 shows three graphs which each illustrate a zoomed-in portion of the graphs shown in FIG. 4, providing more detail regarding the lower-left quadrant.

FIG. 12 shows a chart summarizing the relative amplicon coverage observed in a PCR assay using chimeric primers at different concentration levels (“MP4,” original concentration; and “MP5,” optimized concentration).

FIG. 13 and FIG. 14 are charts analyzing primer dimer formation observed in the MP4 and MP5 test groups, respectively.

FIG. 15 shows three chromatograms analyzing the amplification results when the CFTR and Tetra Chimeric Assays are combined into a single multiplex PCR assay (top), and when the CFTR Chimeric Assay (middle) and Tetra Chimeric Assay (bottom) are run as separate reactions.

FIG. 16 is a chart summarizing primer dimers results when the CFTR and Tetra Chimeric Assays are combined into a single multiplex PCR assay (top), and when the CFTR Chimeric Assay (middle) and Tetra Chimeric Assay (bottom) are run as separate reactions.

FIG. 17 is a graph showing a coverage comparison between the Tetra Chimeric Assay, separately and in combination with the CFTR Chimeric Assay.

FIG. 18 is a graph showing a coverage comparison between the CFTR Chimeric Assay, separately and in combination with the Tetra Chimeric Assay.

### DETAILED DESCRIPTION OF VARIOUS EMBODIMENTS

A “polymerase chain reaction” or “PCR” is an enzymatic reaction in which a specific template DNA is amplified using one or more pairs of sequence specific primers for a single target. A “multiplex polymerase chain reaction” or “multiplex PCR” is an enzymatic reaction that employs two or more primer pairs for different targets templates. If the target templates are present in the reaction, a multiplex polymerase chain reaction results in two or more amplified DNA products that are co-amplified in a single reaction using a corresponding number of sequence-specific primer pairs.

Primer dimer formation is a concern when conducting PCR amplification, as these off-target amplification products divert system resources (e.g., primers, polymerase, and dNTPs) away from the target PCR reactions. This issue is of particular concern in multiplex PCR, where the presence of several, if not many, different primers significantly increases the possibility for unintended cross-reactions and dimer formation. This issue is the major hurdle when building large multiplex PCR assays and also hampers fast turnaround time for smaller multiplex PCR assays. The mitigation of primer dimer formation consequently requires a significant investment of resources, time, and effort and must be done for every assay, resulting in a longer time to market period. Multiplex PCR assays that produce an amplification product which comprising e.g., >5% primer dimers incur increased sequencing costs for customers and can lead to suboptimal results. As such, the development of commercially-viable multiplex PCR assays often requires many optimization rounds and the iterative redesign of primers in order to reduce primer dimer formation to a more acceptable level (i.e., <5%).

Many methods have been proposed to circumvent the non-specific amplification often observed in in multiplex PCR. For example, U.S. Pat. No. 8,460,874 (“Peleg”) discloses that ribonucleotides may be incorporated into standard DNA primers used for multiplex PCR, in order to improve specificity. However, while Peleg suggests that adding RNA bases to a DNA primer reduces non-specific amplification, Peleg also expressly teaches that adjacent ribonucleotides cannot be used to prevent non-specific amplification in general and primer dimer formation in particular. See e.g., Peleg at 3:23-28 (cautioning that “incorporating only a few ribonucleotides in a DNA primer ... can have a beneficial impact on reducing generation of undesired artifacts produced by in-vitro DNA-dependent DNA-polymerase amplification reactions, provided that the ribonucleotides are non-adjacent”). Moreover, Peleg’s approach fails to provide rules that can be consistently applied based solely on the original sequence of a given pair or set of DNA primers (e.g., multiple solutions may be possible and undue experimentation may be necessary to identify useful parameters). Other known techniques for addressing non-specific amplification or primer dimer formation in multiplex PCR have been based on iterative redesign of primers or workflows involving enzymatic steps to remove formed primer dimers or activate blocked primers.

In view of these and other shortcomings of current methods, there exists a need for new methods for primer design. In particular, there exists a need for methods that can be used to more efficiently design standard and multiplex PCR assays that amplify one or more target DNA sequences in a reaction mixture while reducing the formation of non-specific amplification products, such as primer dimers. Such methods, as described herein, rely on the use of at least one “chimeric primer” comprising one or more DNA bases and at least one segment comprising two or more adjacent RNA bases. In some aspects, these chimeric primers may optionally include a DNA base at the 3′ end. In some aspects, the two or more adj acent RNA bases may be incorporated into a chimeric primer at a position that is 5’ of the overlapping region that would result when the unmodified DNA version of the chimeric primer hybridizes to another primer in the reaction mixture under the conditions selected for a given PCR assay.

In some aspects, the disclosure provides chimeric primers which are advantageous in that they can be used to reduce or eliminate the formation of non-specific amplification artifacts and primer dimers without the need for pilot tests to first identify problematic primer pairs in a given reaction mixture, saving time and resources. For example, the introduction of RNA bases into the overlapping region formed by a pair of primers reduced primer dimer formation. However, primers may interact with multiple other primers in a reaction mixture, resulting in different pairings with different overlapping regions. Given that the overlapping region may fluctuate from pair to pair, iterative testing may be required to identify the optimal position for the RNA bases to be included in a given chimeric primer. In contrast, the present methods described herein allow one to design a chimeric primer based solely on the sequence of a DNA primer capable of being used in a PCR assay. No additional information or testing is needed, reducing development time and costs.

The chimeric primers according to any of the exemplary aspects disclosed herein may comprise RNA analogs in place of one or more of the RNA bases described herein. For example, a chimeric primer may comprise at least one segment comprising two or more adjacent RNA analogs. RNA analogs include, e.g., 2′-O-Methyl RNA, wherein a methyl group is added to the 2′ hydroxyl of the ribose moiety. Any other RNA analogs known in the art may be used. In the interests of brevity, the disclosure generally refers to chimeric primers which comprise RNA bases. However, to be clear any such reference is intended to also contemplate an alternative aspect wherein one or more of such RNA bases are replaced by RNA analogs.

Without being bound to a theory or mechanism, it is believed that because a DNA-RNA binding is more stable, chimeric primers will therefore tend to only align to the DNA template in a PCR reaction mixture, preventing the formation of primer dimers. The straightforward primer design rules described herein are amenable to automation and can be used to substantially reduce (if not eliminate) the lengthy process of iterative design and testing required by current methods. As such, the methods described herein can be used to develop and/or optimize PCR assays faster, reducing effort, time, costs, and time-to-market.

### PCR Assays

The disclosure provides PCR methods which use chimeric primers, as well as compositions and kits useful for such methods. In some aspects, a PCR reaction mixture may contain at least one chimeric primer (e.g., two or more chimeric primer pairs). The PCR reaction mixture may also contain nucleotides, e.g., dGTP, dATP, dTTP and dCTP, a DNA polymerase, e.g., a thermostable DNA polymerase, and PCR reaction reagents, which may be a pH-buffered solution containing salt (e.g., MgC1₂) and other components necessary for PCR. In certain embodiments, the PCR reaction mixture may further contain a nucleic acid sample (e.g., comprising genomic DNA and/or mRNAs). In certain embodiments, the components of a PCR reaction may be at a concentration suitable for PCR.

PCR conditions of interest include those well known in the art (e.g., Ausubel, et al., Short Protocols in Molecular Biology, 3^{rd} ed., Wiley & Sons 1995 and Sambrook et al., Molecular Cloning: A Laboratory Manual, 3^{rd} third Edition, 2001 Cold Spring Harbor, N.Y. for example). The amounts of the amplification products may be assessed after any number of rounds of PCR amplification (i.e., successive cycles of denaturation, re-naturation and polymerization). In certain embodiments, the amount of any amplification product may be assessed a stage at which the nucleic acid amplification occurs linearly (i.e., during the linear phase of the amplification reaction) or after the reaction rate has reached a plateau.

If a multiplex PCR mixture including chimeric primers according to the disclosure is used in a PCR method, the method may produce at least 5, at least 10, at least 15, at least 20, at least 25, at least 30, at least 35, at least 40 or at least 50 or more resolvable amplification products. A reaction mixture may be employed in multiplex PCR methods to co-amplify 10 or more products; 50 or more or products; 100 or more products; 250 or more products; 500 or more products; 1,000 or more products; 2,500 or more products; 5,000 or more products; or 10,000 or more products; in certain cases without detectable (or significant) formation of primer dimers. Multiplex PCR methods using chimeric primers according to the disclosure may be employed to amplify at least 1.5 times, at least 2 times, at least 3 times, at least 5 times or at least 10 times the number of target PCR products than an otherwise identical methods that employ only DNA primers.

The results obtained from an assay may be graphed, and, in certain embodiments, the sizes and/or the abundance of the amplification products may be calculated. Similarly, any such products may be sequenced in whole or in part. Any evaluation may be qualitative or quantitative. The PCR amplification methods described herein may be performed using a thermal cycler (e.g., a SureCycler 8800 Thermal Cycler from Agilent Technologies, Inc., a Veriti Thermal Cycler from Thermo Fisher Scientific, or a thermal cycler sold by another manufacturer).

### Chimeric Primers and Methods of Designing the Same

A “chimeric primer” according to disclosure is an oligonucleotide comprising deoxynucleotides and ribonucleotides (also referred to herein as DNA bases and RNA bases, respectively), having a sequence that includes a segment comprising at least two adjacent RNA bases. As is understood in the art, a “primer” is an oligonucleotide that can be extended from its 3′ end by the action of a polymerase as part of an in vivo or in vitro DNA synthesis reaction. An oligonucleotide that cannot be extended from it 3′ end by the action of a polymerase is not a primer. As such, all chimeric primers must be capable of being used to amplify DNA (e.g., as part of a pair of primers capable of amplifying a segment of genomic DNA when used in a PCR reaction).

In some aspects, a chimeric primer according to the disclosure may have a DNA base at the 3′ end. The segment comprising at least two adjacent RNA bases may be located at any position on the chimeric primer. However, in some aspects, this position will be within 1, 2, 3, 4, 5, 6, 7, 8, 9, 10, 11, 12, 13, 14, or 15 bases of the 3′ end of the primer. In some aspects, the chimeric primer may comprise multiple segments which each comprise at least two adjacent RNA bases, wherein any such segments are separated from each other by at least two adj acent DNA bases. For example, a chimeric primer may comprise two or three segments, each comprising two adjacent RNA bases, with two adjacent DNA bases separating any such segments.

In some aspects, a chimeric primer may be a primer that functions as part of a pair with a DNA primer, e.g., one being a forward primer and the other a reverse primer configured to multiply a given amplicon. In some aspects, this primer pair may be configured to multiply the amplicon while reducing or eliminating primer dimers compared to the amplification product produced if a DNA primer equivalent is used in place of the chimeric primer, under otherwise identical PCR assay conditions. In some aspects, the chimeric primers described herein may be used in a PCR assay comprising a multiplex PCR assay that generates an end product comprising less than 1, 2, 3, 4, 5, 6, 7, 8, 9 or 10% primer dimer amplification products, again compared to an otherwise identical PCR assay using DNA primer equivalents is used in place of any such chimeric primers.

In some aspects, a chimeric primer may comprise an oligonucleotide wherein the at least two RNA bases comprise a pair of RNA bases located at either positions 7 and 8, or 14 and 15, as measured from the 3′ end of the chimeric primer. In other aspects, the chimeric primer may comprise a pair of RNA bases located at positions 2 and 3, 3 and 4, 4 and 5, 5 and 6, 6 and 7, 8 and 9, 9 and 10, 10 and 11, 11 and 12, 13 and 14, 15 and 16, 16 and 17, 17 and 18, 18 and 19, 19 and 20, or any combinations thereof.

In some aspects, a chimeric primer capable of amplifying DNA while minimizing or eliminating non-specific amplification may be generated by a method, comprising: a) identifying a non-specific amplification fragment (e.g., a primer dimer) produced during a PCR assay; b) identifying one or more DNA primers that produce the non-specific amplification fragment; and c) selecting a sequence for at least one chimeric primer; wherein the chimeric primer is an oligonucleotide comprising DNA and RNA bases, and has a sequence identical to that of one of the identified DNA primers except for a first segment wherein at least two adjacent DNA bases are replaced by corresponding RNA bases. In some aspects, a sequence for the chimeric primer may be selected based upon the hybridization of the one or more primers which produce the non-specific amplification product. For example, a given pair of primers may be found to generate a particular primer dimer. This pair of primers may be analyzed (e.g., in software) to determine the extent to which these primers overlap under the PCR assay conditions and parameters in which they will be used. Once the overlapping region is identified, one or more segments on either of the DNA primers, 5′ of the overlapping region, may be selected for replacement with RNA bases in order to generate a chimeric primer. This process may be applied to either or both DNA primers, and is illustrated by an example below. Chimeric primers generated using the methods described herein may be used for standard or multiplex PCR, and may be provided e.g., in kits for performing PCR.

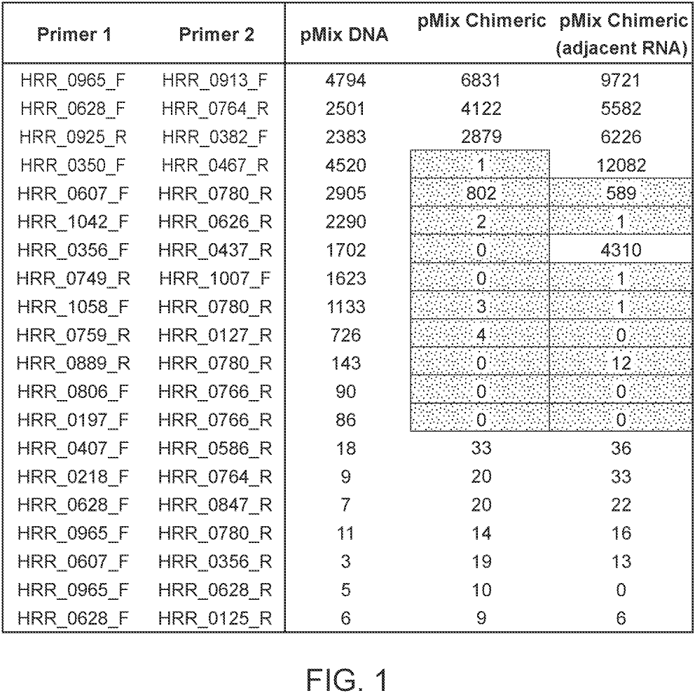

The inclusion of at least one chimeric primer in a PCR assay normally reduces the formation of primer dimers. FIG. 1 illustrates the results of a series of PCR assays using standard DNA primers, chimeric primers produced according to Peleg’s disclosure, and chimeric primers according to the present disclosure (with two adjacent RNA bases, contrary to Peleg’s approach). The protocol and parameters of this study are described in further detail below as “Experiment #2”. Shaded boxes highlight primer pairs which resulted in primer dimer formation where at least one of the DNA primers was replaced by a chimeric primer. This data confirms that PCR assays using chimeric primers according to the present disclosure generated far fewer primer dimers than assays using standard DNA versions of these same primers. Moreover, the chimeric primers produced according to the present disclosure displayed results that are comparable to, and in some cases better than, Peleg’s chimeric primers. This outcome is surprising and unexpected because chimeric primers produced according the present disclosure include adjacent RNA bases in direct contradiction to Peleg’s rule that such configurations are unusable.

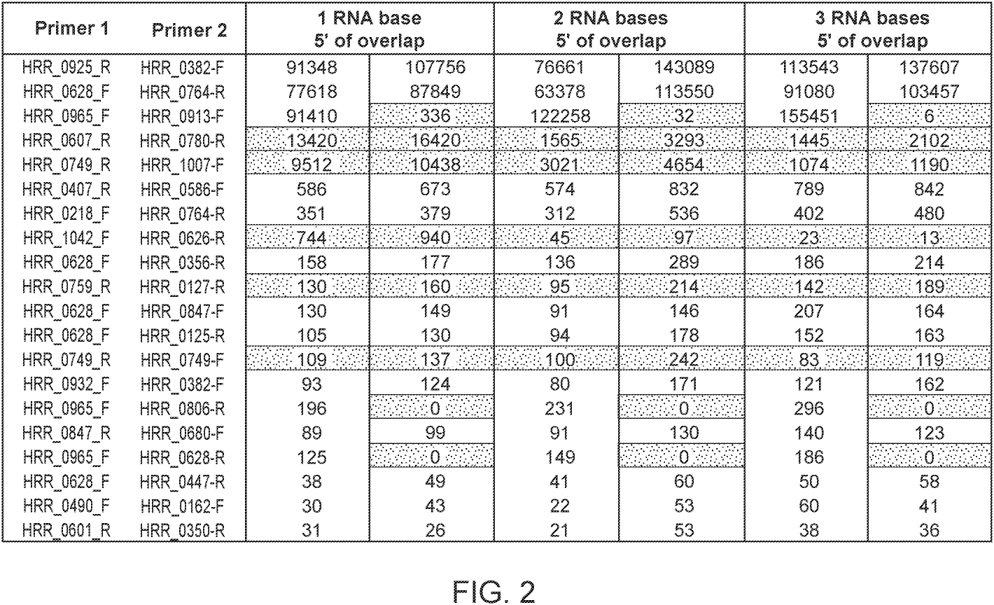

Further experiments were performed to study the impact of incorporating multiple adjacent RNA bases into at least one primer used in a PCR assay. As illustrated by FIG. 2, several chimeric primers were generated by replacing 1, 2, or 3, adjacent DNA bases of a standard DNA primer, with corresponding RNA bases. These RNA bases were incorporated at a position 5′ of the overlapping region produced when the unmodified primer hybridizes two the other member of its primer pair under standard PCR assay conditions. The protocol and parameters of this study are described in further detail below as “Experiment #3”. Shaded rows denote tested primer pairs where at least one primer in the assay was a chimeric primer (i.e., unshaded rows represent pairs of unmodified DNA primers). The results of this study indicate that chimeric primers which include two or three RNA bases tend to be more effective than chimeric primers having a single RNA base. This study also illustrated that suppressing certain primer pairs does not result in other primer pairs forming new or additional primer dimers, highlighting the usefulness and applicability of the present methods to multiplex PCR assays (which include several, if not many, primer pairs).

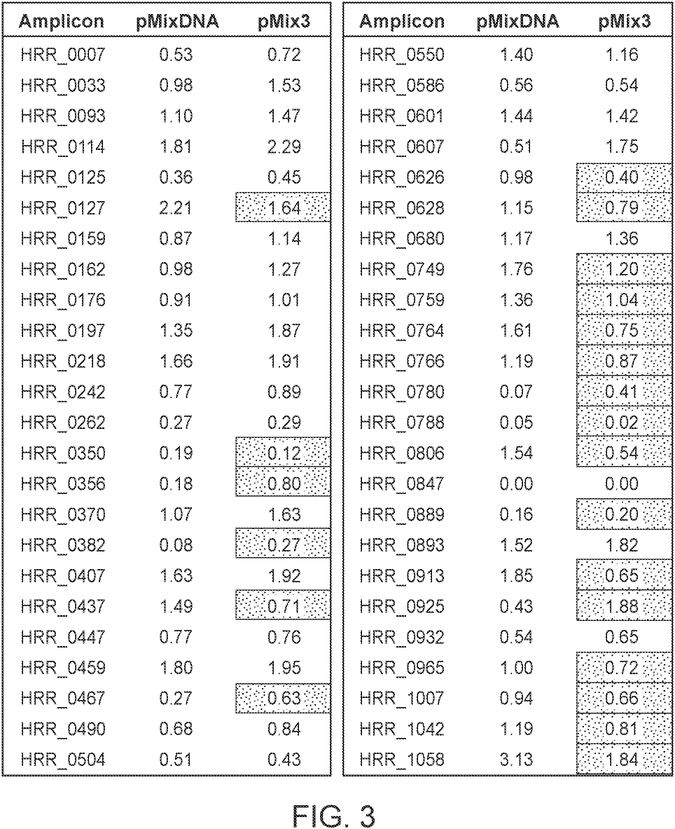

FIG. 3 summarizes the results of another study that examined whether the conversion of DNA primers to chimeric primers affects amplicon yield. The protocol and parameters of this study are described in further detail below as “Experiment #4”. In this case, a multiplex PCR assay was performed using a variety of pairs of DNA primers. Amplicon coverage results for this assay are provided in the column entitled “pMixDNA.” This PCR assay was repeated, with several of the primers converted to chimeric primers in accordance with the present disclosure, with amplicon coverage results provided in the column entitled “pMix3.” Shaded boxes denote primer pairs that include at least one chimeric primer. As illustrated by this figure, the relative coverage of most of the tested amplicons was not significantly impacted by the conversion to chimeric primers.

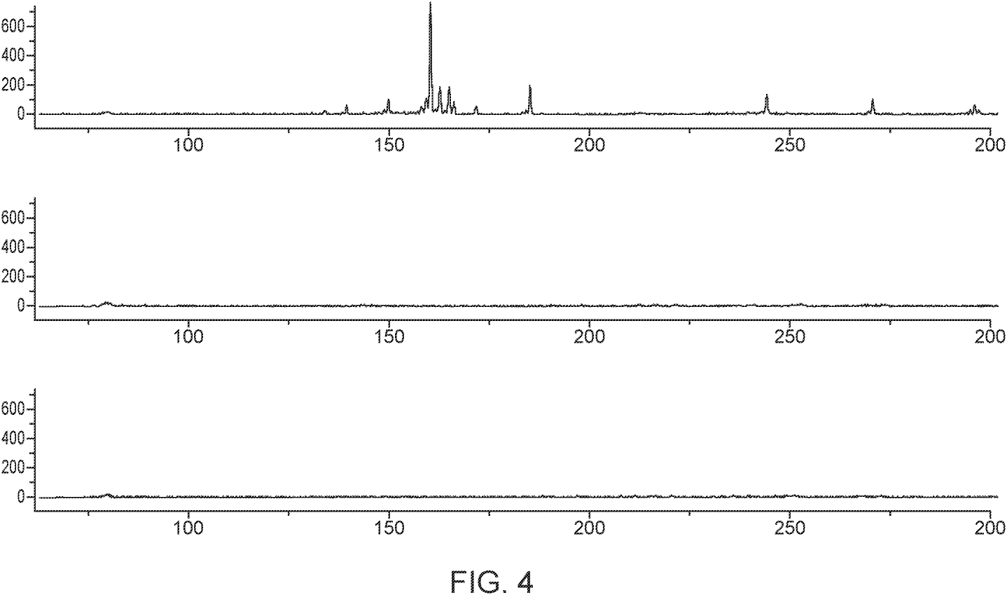

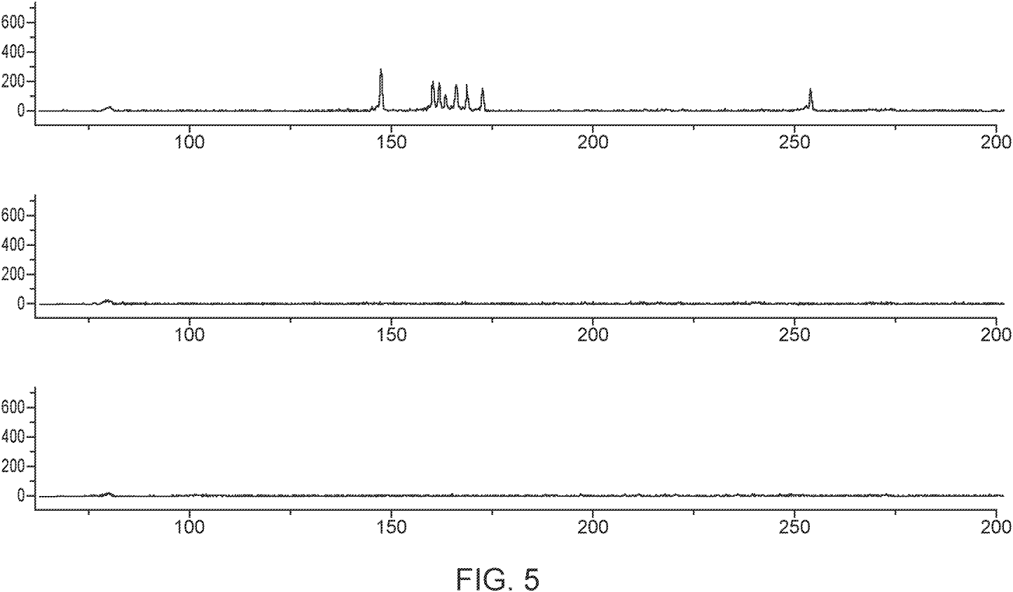

In general, the use of chimeric primers for PCR amplification increases specificity and reduces the formation of non-specific fragments, as evidenced by FIGS. 4 and 5. FIG. 4 includes three chromatograms (Genescans) showing the results of a PCR assay that were conducted using: universal DNA primers on genomic DNA (top); and repeated with two different sets of universal chimeric primers (middle, bottom). The universal DNA primers should not have a complement in the genome, but nevertheless non-specific fragments were formed (i.e., as confirmed by the multiple peaks in the top chromatogram). The middle and bottom chromatograms show no peaks, indicating that non-specific fragments were not detected, and thus confirming the increased specificity of the chimeric primers tested. All chimeric primers tested in this assay had at least one group of 2 adjacent RNA bases. FIG. 5 includes a set of three chromatograms summarizing the results of a related study, which compared the amplification product produced by a 30-cycle PCR using universal DNA primers (top) or universal chimeric primers (middle, bottom). Non-specific fragments were detected solely in the product generated by the DNA primers, once again confirming that chimeric primers produced according to the present disclosure outperform standard DNA primers.

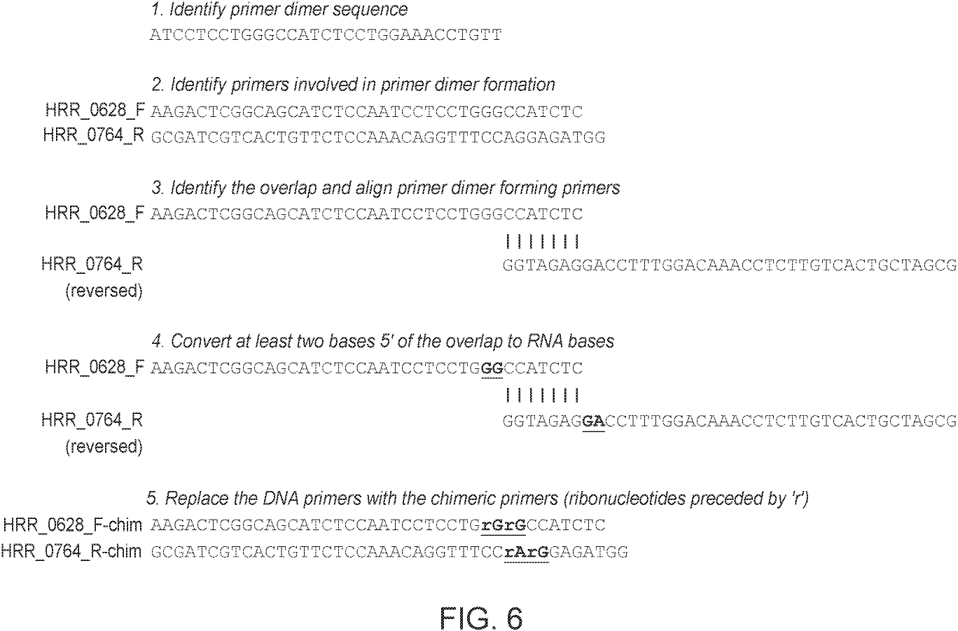

FIG. 6 depicts an exemplary workflow for generating chimeric primers according to the disclosure, highlighting the location of the overlapping region between this representative pair of primers. This figure illustrates that the process may begin with the identification of a primer dimer sequence. Once a dimer sequence is identified, the pair of primers that generated this primer can be identified by analyzing the primers pairs included in the standard or multiplex PCR assay that generated the primer dimer sequence. Primer dimers are formed during a PCR amplification as a result of the unintended hybridization of at least two primers included in the reaction mixture. As such, once a set of at least two primers associated with dimer formation are identified, the method can proceed by identifying the overlapping region between at least two primers in the set, as shown by step 3 in this figure. In some aspects, this overlapping region may be identified by determining a pairwise alignment of the selected primer sequence under conditions used for an intended PCR assay (e.g., accounting for the salt concentration, temperature parameters, etc. that will be used). The overlapping region may also be determined using computational modeling (e.g., using a molecular dynamic simulation). After the overlapping region is determined, chimeric primer versions of either (or both) of the original DNA primers can be generated by selecting at least two adjacent deoxynucleotides 5′ of the overlapping region and converting these deoxynucleotides to ribonucleotides. In this example, steps 4 and 5 illustrate the selection and conversion of the two deoxynucleotides immediately adjacent to the overlapping region, on both primer sequences. It is understood that any of the steps of this chimeric primer design process (except for the final synthesis of a primer molecule) may be performed using software. In particular, it is envisioned that the present methods may be included in an automated process for rapidly designing and optimizing the set of primers used in a multiplex PCR assay.

In some aspects, a chimeric primer capable of amplifying DNA while minimizing or eliminating non-specific amplification may be generated by a method, comprising: a) selecting a DNA primer from a pair of DNA primers configured to amplify at least a portion of a template DNA molecule in a PCR assay; b) selecting a sequence for a chimeric primer, wherein the chimeric primer is an oligonucleotide comprising DNA and RNA bases, having a sequence that includes a first segment consisting of two adjacent RNA bases, said sequence being identical to the sequence of the DNA primer selected in step a) except for the first segment; and c) optionally, generating the chimeric primer (e.g., by any known oligonucleotide synthesis method). In some aspects, the first segment spans positions 3 and 4, 5 and 6, 6 and 7, 7 and 8, 8 and 9, or 14 and 15, as measured from the 3′ end of the chimeric primer. In other aspects, the first segment may span any two adjacent bases of the chimeric primer.

Chimeric primers designed according to the present methods may be used to amplify DNA in a PCR assay. For example, such methods may comprise conducting a PCR assay using a reaction mixture comprising one or more chimeric primers, a DNA-dependent polymerase, and a template DNA molecule; and b) amplifying at least one segment of the template DNA molecule using the one or more chimeric primers; wherein each of the one or more chimeric primers is an oligonucleotide comprising DNA and RNA bases, having a sequence that includes a first segment comprising at least two adjacent RNA bases.

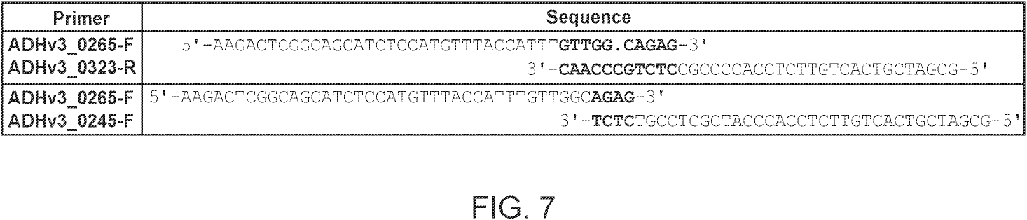

Chimeric primers produced according to the disclosure may be advantageously generated without the need for prior tests to identify problematic primer pairs (e.g., pairs which form primer dimers). Moreover, as noted above primers can interact with multiple other primers in a given reaction mixture, resulting in multiple pairings having different overlapping regions. The existence of these multiple pairings complicates the identification or problematic primer pairs and overlapping regions that could be targeted for conversion to RNA bases. FIG. 7 shows an example of this problem, where the overlap between the primers is highlighted. Primer ADHv3_0265-F is involved in both primer dimers, in one case with an overlap of 10 bp, and another with only 4 bp. Assuming that both primer dimers make up more than 15 % of the total primer dimers in a given PCR assay, both should be avoided. However, this scenario would require designing and testing of two possible chimeric primers for ADHv3_0265-F, if one is using a chimeric primer design rule that requires information about the overlapping region.

Option 1:

AAGACTCGGCAGCATCTCCATGTTTACCATrUrUGTTGGCAGAG

Option 2:

AAGACTCGGCAGCATCTCCATGTTTACCATTTGTTGrGrCAGAG

One would then have to test both chimeric primers in separate PCR assays to determine which performs better, wasting resources and time. This troubleshooting and refinement process can often require significant resources in the case of a large multiplex PCR assay which includes numerous primers.

In contrast, the chimeric primer design rules disclosed herein may be applied to eliminate the need for the iterative testing and refinement process, as they only require information about the sequence of an initial DNA primer to be converted into a chimeric primer. A pair of adj acent DNA bases at specific positions along the sequence of the DNA primer may be converted to their corresponding RNA bases, reducing the formation of primer dimers while maintaining amplicon coverage.

### Example #1: Evaluation of Chimeric Primers Designed Without Prior Information Regarding Dimer Formation

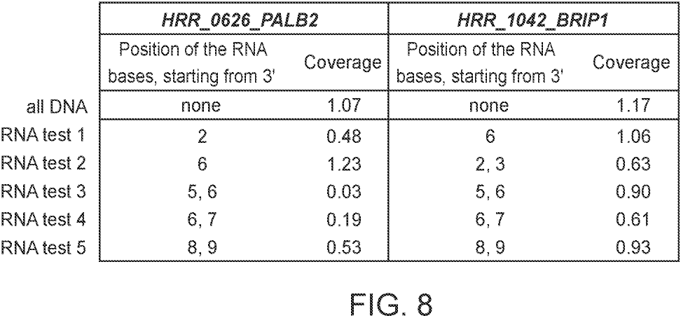

FIG. 8 summarizes the results of a study which examined the effect of converting DNA bases at 5 fixed positions to RNA bases, gradually moving from the 3′ end to the 5′ end of the chimeric primer. As shown by FIG. 8, shifting the pair of adjacent RNA bases more towards the 5′-end, has an effect on the yield.

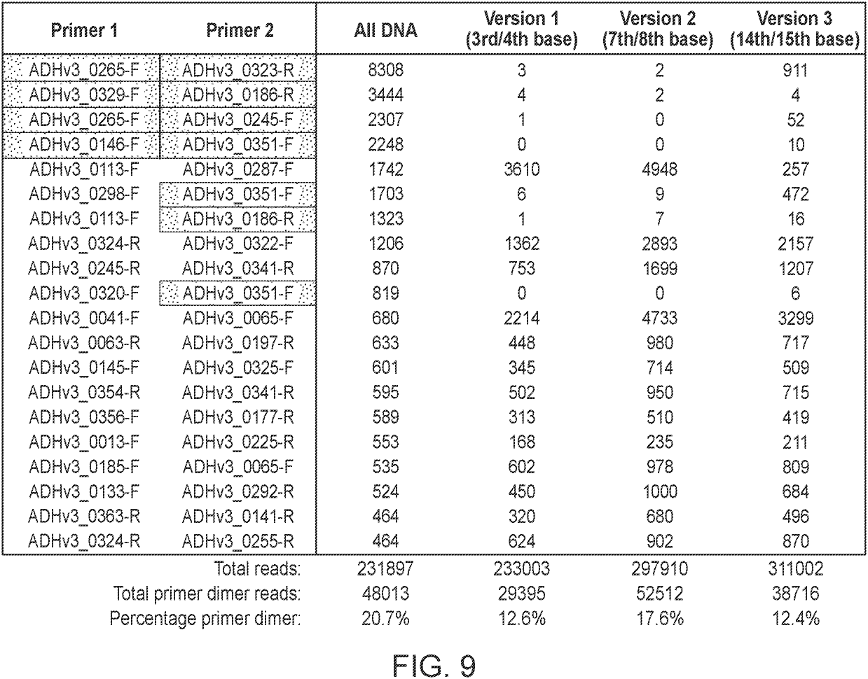

Based on these results, new primers were designed, with RNA bases placed at three fixed positions: the 3^{rd} and 4^{th} base, starting from the 3′-side (“Version 1”); the 7^{th} and 8^{th} base, starting from the 3′-side (“Version 2”); the 14^{th} and 15^{th} base, starting from the 3′-side (“Version 3”). These chimeric primers were tested in a PCR assay to analyze the levels of primer dimers formed by these chimeric primers compared to standard DNA primers (all chimeric primers are noted with shading). The results of this study are summarized on the chart shown in FIG. 9. As illustrated by FIG. 9, shifting the RNA bases further away from the 3′-side (Version 3) still reduces the amplification of primer dimers, but less so than the other two versions. For most of the tested primers, there is not much difference between Version 1 and Version 2 in reducing the level of primer dimers. As such, chimeric primers having a pair of adjacent RNA bases at positions 3 and 4, or 7 and 8, were shown to be particularly effective in reducing the level of primer dimer formation in this exemplary set of primers.

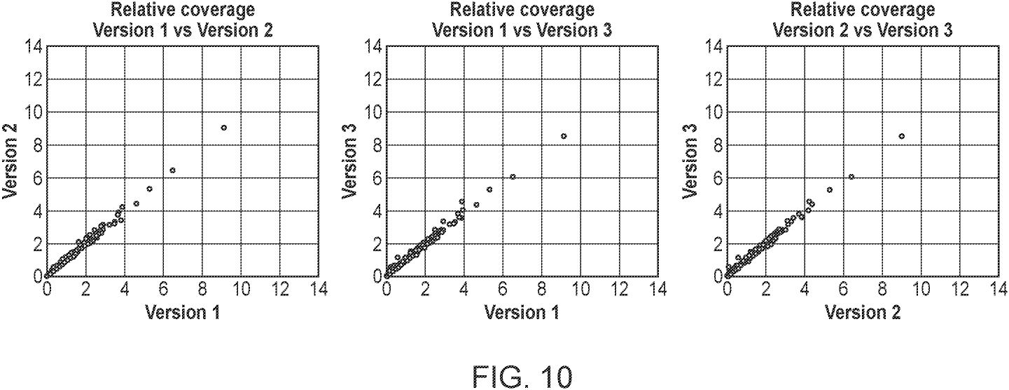

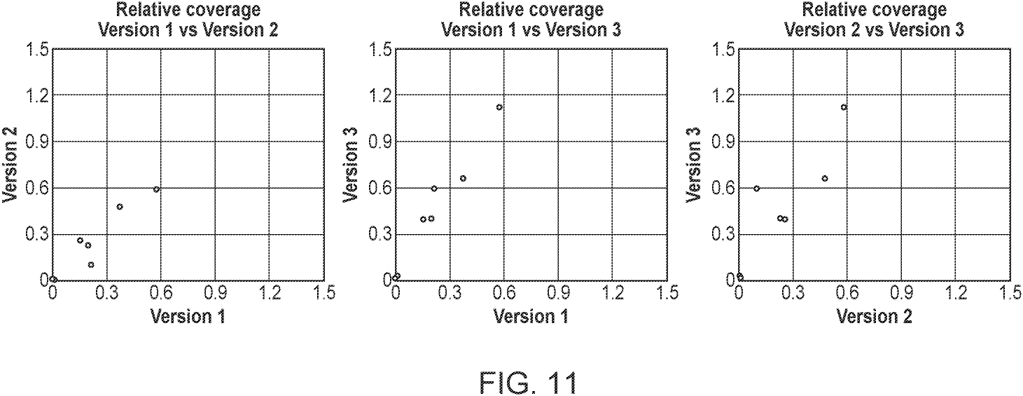

The chimeric primers examined in this study were further examined to analyze amplicon coverage. The coverage of the regular amplicons is not affected by the position of the RNA base. As illustrated by the graph provided as FIG. 10, the overall coverage of all primers in the three different primer mixes is almost identical. When zooming in on the coverage of the chimeric primers (FIG. 11) it becomes clear that the coverage of these chimeric primers in Versions 1 and 2 is more or less identical, but the coverage in Version 3 is a bit higher. Without being bound to a theory, the RNA bases are further away from the 3′-end in Version 3, and it appears that their effect on the aligning and amplification decreased, which decreases the effect on primer dimer formation, but no longer reduces the efficiency of the amplification.

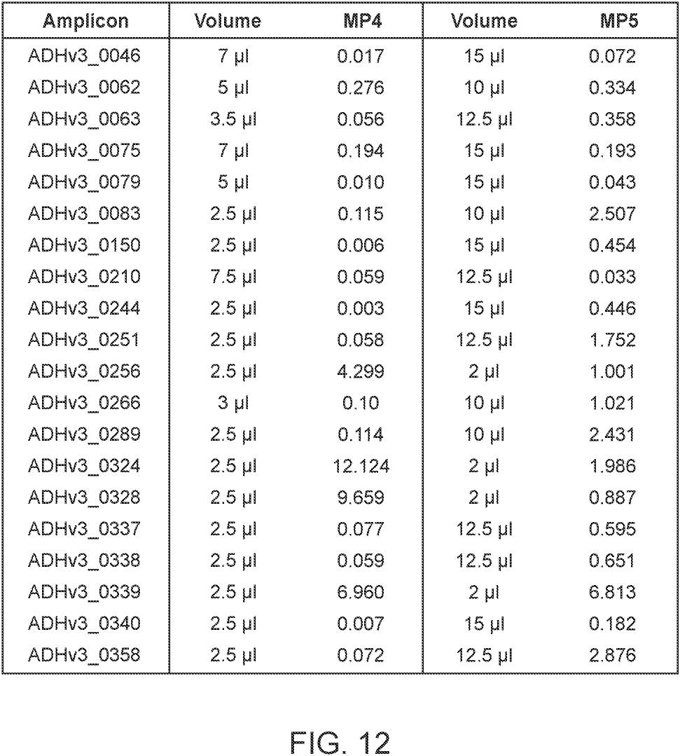

Multiplex PCR assays are becoming increasingly common, and the chimeric primers of the present disclosure are well-suited for such assays. To improve the number of samples the customer can handle per run, the relative coverage of each amplicon in an assay must be relatively equal. Not every primer has the same efficiency, so concentrations in the primer mix must be optimized. To do so in an all-DNA assay, the volume (and thus concentration) of each primer is adjusted individually until the predetermined specifications are met. The same approach can be applied in assays with chimeric primers. In order to evaluate the use of chimeric primers in a representative multiplex PCR assay, the entire ADHv3 MASTR assay was prepared with 2 RNA bases at positions 7 and 8 from the 3′ end for all primers. This assay was performed using the original setup with unmodified volumes (MP4) and as a second run with optimized volumes (MP5). FIG. 12 is a chart showing relative amplicon coverage for a representative set of primers that were included in the MP4 and MP5 assays. Some of the primers are not affected by the concentration changes (e.g., ADHv3_0062, ADHv3_0075) while others react very well (e.g., ADHv3_0083, ADHv3_0358). The few primers that do not react at all, may be redesigned (e.g., ADHv3_0079, ADHv3_0210). Primers with decreasing volumes (e.g., ADHv3_0256, ADHv3_0324) also decrease in coverage. As illustrated by these results, an all-chimeric primer assay can be optimized in the same way as standard, all-DNA assays, i.e., by simply changing the concentration of the primer(s).

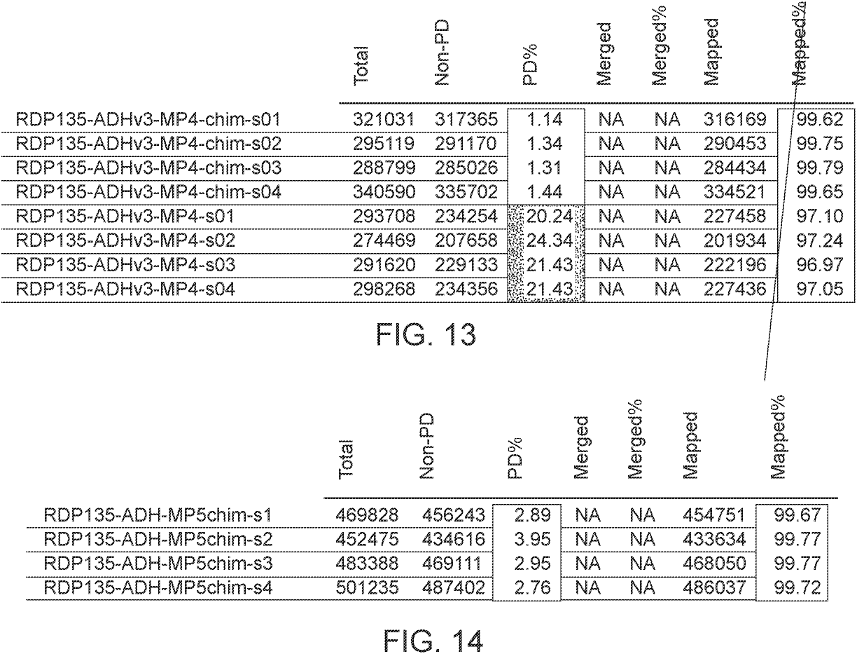

The amplification products produced by the MP4 and MP5 assays were sequenced to analyze primer dimer levels. FIG. 13 shows the result of the primer dimer analysis after sequencing. The first 4 samples were run on the chimeric primer assay, and the next 4 samples were run on the DNA primer assay. The drop in primer dimers when using chimeric primers is clear, with the level of primer dimers dropping from > 20% in the DNA primer setup to < 1.5% in the chimeric primer setup. After optimizing the primer concentrations, the primer dimer analysis of MP5 (FIG. 14) shows a small increase in primer dimer levels compared to MP4, but this level still much lower than in the all-DNA primer mix (e.g., dropping to approximately 2-4% versus >20%).

As illustrated by this exemplary data, an all-chimeric primer assay with RNA bases at fixed positions by design has a significant positive effect on the primer dimer generation (e.g., a 20-fold decrease in primer dimer formation). Additionally, as the chimeric primers bind more specific to their target, they will amplify less off-target fragments (e.g., as evidenced by the “Mapped %” column in the charts provided as FIGS. 13 and 14). As a result, the chimeric primers and design methods disclosed herein may be used to efficiently design chimeric primers, avoiding the slow and costly iterative design process required by known methods.

### Example #2: Reduction of Primer Dimers in a Multiplex PCR Assay Using Chimeric Primers Designed Based on Information Regarding Primer-Dimers

A first multiplex PCR amplification assay (labeled, “RDP135-5-pMixDNA”) was conducted using a reaction mixture comprising genomic DNA, a set of DNA primers configured to amplify multiple amplicons, and a PCR reaction mixture (containing deoxynucleotides, a thermostable DNA polymerase, a pH-buffered solution containing MgC1₂, and other components necessary for PCR) and tested on two samples (“s1” and “s2”). This assay functioned as control group in this study. In particular, the first PCR reaction included a set of specific primers (0.5 µM per primer), 250 µM of each dNTP, 1x Titanium Taq buffer, Taq polyemerase, and 3.5 mM MgC1₂. The PCR proceeded at 98° C. for 10 minutes to denature the template, followed by 20 rounds of amplification, cycling between 95° C. (45 seconds, denaturation), 60° C. (45 seconds, annealing), and 68° C. (2 minutes, extension), before finally shifting to 72° C. (10 minutes). The second PCR on these samples included a set of universal PCR primers (0.5 µM per primer) rather than specific primers, but followed an otherwise identical protocol, except that the annealing step was performed at 64° C.

A second multiplex PCR amplification assay (labeled, “RDP135-5-pMix3”) was conducted using the same conditions and parameters, except for the replacement of a subset of the DNA primers with chimeric primers prepared in accordance with the present disclosure and tested on the same two samples. In particular, both samples were analyzed to identify pairs of DNA primers which generate primer dimers, and each of these problematic DNA primers was replaced with a corresponding chimeric primer which incorporated a pair of adjacent ribonucleotides 5′ of the overlapping region generated when these DNA primers hybridize with each other. DNA primers in both sets which were found not to generate primer dimers were left unmodified.

The amplification products generated by these four PCR samples were collected and sequenced. The results of this analysis are summarized on Table 1 below.

TABLE 1

A comparison of primer dimer formation using DNA primers vs. chimeric primers

| | Total Reads | Primer Dimer Reads | Primer Dimer (%) |
|---|---|---|---|
| RDP13 5-5-pMixDNA-s1 | 87,973 | 67,576 | 76.81 |
| RDP135-5-pMix3-s1 | 97,149 | 1,154 | 1.18 |
| RDP 13 5-5-pMixDNA-s2 | 82,277 | 60,490 | 73.51 |
| RDP135-5-pMix3-s2 | 92,828 | 1,447 | 1.55 |

As illustrated by Table 1, the replacement of problematic DNA primers in the S1 and S2 sets, with corresponding chimeric primers, resulted in a substantial reduction in the amount of primer dimer in both cases (i.e., from >70% down to approximately 1%).

### Example #3: A Comparison of DNA Amplification Using Primers Designed According to Peleg’s Rules Versus Primers Designed According to the Present Disclosure

An experiment was conducted to compare the performance of primers designs according to Peleg’s design rules (e.g., having a plurality of non-adjacent ribonucleotide substitutions) versus primers designed according to the present disclosure, which have at least two adjacent ribonucleotide substitutions. The results of this experiment are summarized in the chart illustrated by FIG. 1.

In this experiment, PCR amplifications were performed using genomic DNA as a template, using the PCR protocol set forth above, except for the structure of the primer pairs used for amplification. A control group (labeled as the “pMix DNA” group) included pairs of DNA primers (e.g., “HRR 0965_F” paired with “HRR_0913 _F”). A first experimental group used variants of the control group primer pair with at least two non-adjacent ribonucleotides, in accordance with Peleg’s design rules (labeled as the “pMix Chimeric (Peleg)” group). A second experimental group used variants of the control group primer pair with at least two adjacent ribonucleotides, in accordance with the present disclosure (labeled as the “pMix Chimeric (adjacent RNA)” group).After the second PCR amplification, the resulting product was collected using AMPure® bead-purification kit. The amplicon library was diluted to 4 nM by spectroscopic measurement and sequenced using an Illumina® MiSeq, system according the manufacturer’s standard protocol.

This study confirms that PCR assays using chimeric primers according to the present disclosure generated far fewer primer dimers than assays using standard DNA versions of these same primers. Moreover, the chimeric primers produced according to the present disclosure displayed results that are comparable to, and in some cases better than, Peleg’s chimeric primers.

### Example #4: A Comparative Analysis of the Impact of the Number of Adjacent RNA Bases Used in Chimeric Primers

An experiment was performed to study the impact of incorporating multiple adjacent RNA bases into at least one primer used in a PCR assay. As illustrated by FIG. 2, chimeric primers were generated by replacing 1, 2, or 3 adjacent DNA bases of standard DNA primers, with corresponding ribonucleotide bases. As indicated on this figure, the ribonucleotide bases were located 5′ of the overlapping region of each tested primer pair. A series of PCR amplifications were performed using the primer pairs shown on FIG. 2, under otherwise identical conditions except for the structure of the primers. This PCR assay followed the same protocol set forth in [0046] above.

The results of this study indicate that chimeric primers which include two or three RNA bases tend to be more effective than chimeric primers having a single RNA base. This study also illustrates that suppressing certain primer pairs does not result in other primer pairs forming new or additional primer dimers.

### Example #5: Amplicon Coverage Studies

Studies were conducted to analyze whether chimeric primers according to the disclosure have an impact on amplicon coverage. In this study, a multiplex PCR assay was performed using a set of multiple pairs of DNA primers (a control group labeled as “pMix DNA”). This PCR assay followed the same protocol set forth above. This PCR assay was repeated, with several of the primers converted to chimeric primers in accordance with the present disclosure (i.e., “pMix3”). The amplified product was collected and sequenced in order to assess the amplicon coverage provided by the tested primer sets. Sequencing confirmed that the relative coverage of most of the tested amplicons was not significantly impacted by the conversion to chimeric primers, as illustrated by the results shown in FIG. 3.

### Example #5: Combination Assays Using Chimeric Primers

As explained above, chimeric primers may be used to reduce or eliminate the formation of primer-dimers, allowing for the use of a larger panel of primer pairs in an assay. In this experiment, three multiplex PCR assays using chimeric primers were evaluated separately and in combination as pairs. In this case, the chimeric primers had RNA bases substituted at position 7 and 8 from the 3′ end. These combined assays are only feasible due to the substantial reduction in primer-dimers provided by the chimeric primers used in this study. In brief, four samples of the I-0092 DNA panel (50 ng/µl) were analyzed. Assays were run, both separate and combined as described below, following the OnePlex MASTRplus protocol. The volumes of the primer mixes used in the combined setup was equal to the assay in the single setup. The universal PCR was purified and analyzed on GeneScan. A library was prepared and evaluated on MiSeq.

Two multiplex PCR assays were tested, alone and in combination: (1) the CFTR Chimeric Assay, which consists of 275 amplicons with an average length of 220 bp; and (2) the Tetra Chimeric Assay, which is a 15 amplicon assay designed for sample authentication targeting 15 SNPs, with an average amplicon length of 152 bp.

Experiments were set up in which these assays were run individually and combined as a single assay. See Table 2 below for the composition of the multiplex PCR reaction. After one purification (1/1.7 ratio) and a 250x dilution, a universal PCR was performed. The universal PCR was purified once (1/1.1 ratio) and analyzed using Applied Biosystems’ GeneScan Analysis Software. The concentration of the reactions was measured using a DropSense system and an equimolar mix of all samples was prepared. The resulting library was analyzed using an Illumina MiSeq system.

TABLE 2

Composition of the multiplex PCR reaction

| | |
|---|---|
| PCR Mix Component | Amount |
| dNTP | 0.5 µl |
| buffer | 5.0 µl |
| Taq polymerase | 0.5 µl |
| CFTR Primer Mix | 10.0 µl |
| Tetra Primer Mix | 6.7 µl |
| DNA (50 ng/µl) 1-0092 panel | 1.0 µl |
| water | Add up to 50 µl |

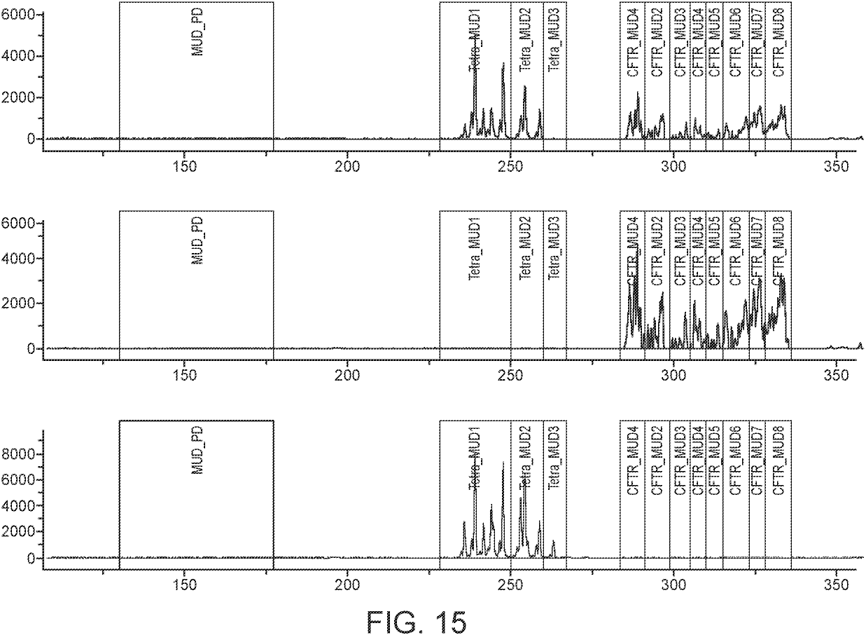

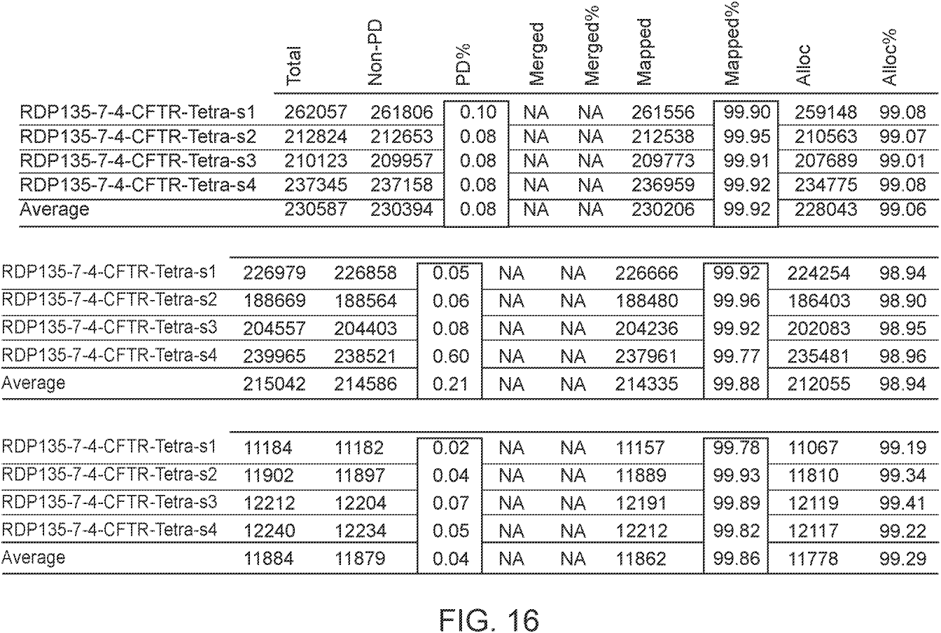

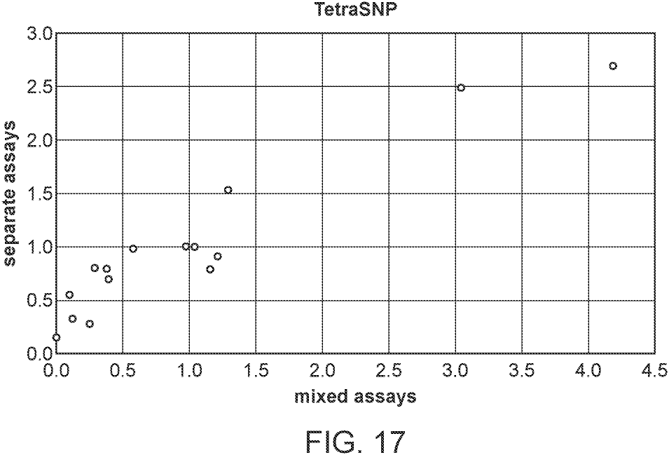

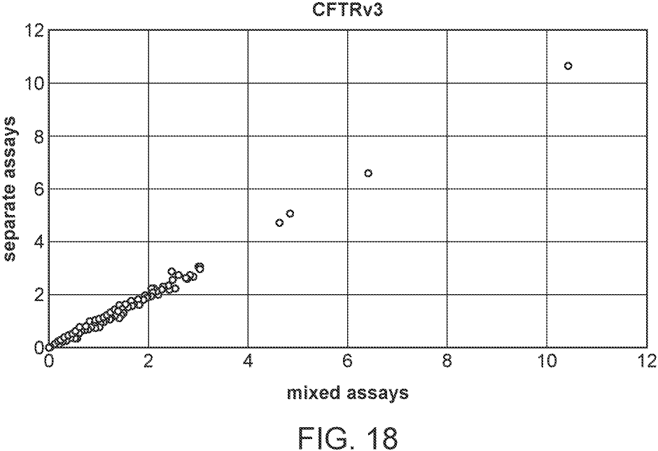

As illustrated by FIG. 15, an analysis of the universal PCR using GeneScan shows good amplification levels and absence of primer dimers for both the separate (bottom two rows) and combined (top row) assays. Similarly, both the CFTR and Tetra Chimeric Assays demonstrate low primer dimer formation. As a standalone assay, the CFTR Chimeric Assay averages at 0.21%, and the Tetra Chimeric Assay averages at 0.04%. When the two assays are combined, primer dimer formation remains low (0.08%), as illustrated by FIG. 16, which shows results for the combined assay (top) and the CFTR Chimeric Assay (middle) and Tetra Chimeric Assay (bottom) as separate reactions. The combined assay boasts 99.92% mapped reads, which is in line with the single assays. Amplicon coverage in the combined assay was also good, with only one Tetra Chimeric Assay amplicon dropping out. FIG. 17 shows a coverage comparison between the Tetra Chimeric Assay as a separate assay and in the combined assay. FIG. 18 shows a coverage comparison between the CFTR Chimeric Assay as a separate assay and in the combined assay.

This study demonstrates that multiplex PCR assays using chimeric primers may potentially be combined for form a larger single assay that provides diagnostic information as to multiple conditions or diseases. Combined assays of this type are made possible due to the substantial reduction in primer dimer formation observed when chimeric primers according to the disclosure are used in place of standard DNA primers.

All statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

1. A method for generating chimeric primers capable of amplifying at least a portion of a template DNA molecule, while minimizing or eliminating the formation of primer dimers, comprising:
a) identifying a primer dimer produced during a PCR assay;
b) identifying one or more DNA primers that produce the primer dimer;
c) identifying an overlapping region produced when the one or more DNA primers hybridize during the PCR assay; and
d) selecting a sequence for at least one chimeric primer; and
e) optionally, generating the at least one chimeric primer;
wherein each chimeric primer is an oligonucleotide comprising DNA and RNA bases, and has a sequence identical to that of one of the identified DNA primers except for a first segment wherein at least two adjacent DNA bases are replaced by corresponding RNA bases.

2. The method of claim 1, wherein the first segment is located outside of the overlapping region or within 15 bases of the 3′ end of the selected sequence, said sequence having a DNA base at its 3′ end.

3. The method of claim 1, wherein the overlapping region is determined based on annealing conditions compatible with a PCR assay.

4. (canceled)

5. The method of claim 1, wherein the selected sequence for the at least chimeric primer further comprises a second segment comprising at least two adjacent RNA bases that replace corresponding DNA bases in the identified DNA primer, wherein the first segment and the second segment are separated by at least two DNA bases.

6. The method of claim 5, wherein the first segment and the second segment are located within 15 bases of the 3′ end of the sequence of the selected sequence or wherein the sequence of the at least one chimeric primer further comprises a third segment comprising at least two adjacent RNA bases that replace corresponding DNA bases in the identified DNA primer, wherein the second segment and the third segment are separated by at least two DNA bases.

7. (canceled)

8. The method of claim 1, wherein selecting a sequence for the at least one chimeric primer comprises:
selecting a sequence for a first chimeric primer configured to hybridize to an upstream portion of the template DNA molecule; and
selecting a sequence for a second chimeric primer configured to hybridize to a downstream portion of the template DNA molecule;
wherein the first and second chimeric primers are capable of amplifying at least a portion of the template DNA molecule by PCR.

9. The method of claim 8, wherein the first chimeric primer and the second chimeric primer each comprise a sequence having a DNA base at its 3′ end, and wherein the first segment of each sequence is located within 15 bases of its respective 3′ end or wherein at least one of the first chimeric primer and the second chimeric primer further comprises a second segment comprising at least two adjacent RNA bases, wherein the first segment and the second segment are separated by at least two DNA bases.

10. (canceled)

11. The method of claim 9, wherein both the first chimeric primer and the second chimeric primer each further comprise a second segment comprising at least two adjacent RNA bases, wherein the first segment and the second segment are separated by at least two DNA bases.

12. The method of claim 1, wherein the at least one chimeric primer is configured to amplify at least a portion of the template DNA molecule while reducing or eliminating formation of non-specific amplification products,
wherein the at least one chimeric primer is configured to amplify at least a portion of the template DNA molecule while reducing or eliminating primer-dimers and/or off-target amplification, or
wherein the PCR assay is a multiplex PCR assay that generates an end product comprising:
a) less than 5% primer dimer amplification products;
b) less than 7% primer dimer amplification products; or
c) less than 10% primer dimer amplification products.

13. (canceled)

14. (canceled)

15. A method for generating chimeric primers capable of amplifying DNA while minimizing or eliminating non-specific amplification, comprising:
a) identifying a non-specific amplification fragment produced during a PCR assay;
b) identifying one or more DNA primers that produce the non-specific amplification fragment; and
c) selecting a sequence for at least one chimeric primer;
wherein the chimeric primer is an oligonucleotide comprising DNA and RNA bases, and has a sequence identical to that of one of the identified DNA primers except for a first segment wherein at least two adjacent DNA bases are replaced by corresponding RNA bases.

16. The method of claim 15, wherein the corresponding RNA bases are located within 15 bases of the 3′ end of the sequence, and the sequence has a DNA base at its 3′ end or wherein the sequence of the chimeric primer further comprises a second segment comprising at least two adjacent DNA bases replaced by corresponding RNA bases, wherein the first segment and the second segment are separated by at least two DNA bases.

17. (canceled)

18. The method of claim 1, wherein at least one of the RNA bases of the chimeric primer is replaced by an RNA analog or wherein the RNA bases of the chimeric primer is replaced by 2′-O-Methyl RNA.

19. (canceled)

20. A method for amplifying DNA, comprising:
a) conducting a polymerase chain reaction (PCR) assay using a reaction mixture comprising one or more chimeric primers, a DNA-dependent polymerase, and a template DNA molecule; and
b) amplifying at least one segment of the template DNA molecule using the one or more chimeric primers;
wherein each of the one or more chimeric primers is an oligonucleotide comprising DNA and RNA bases, having a sequence that includes a first segment comprising at least two adjacent RNA bases.

21. The method of claim 20, wherein at least one of the chimeric primers has a sequence consisting of a) DNA bases and b) at least one pair of adjacent RNA bases spanning positions 3 and 4, 7 and 8, and/or 14 and 15, as measured from the 3′ end of the chimeric primer,
wherein the sequence of at least one of the chimeric primers further comprises a second segment comprising at least two adjacent RNA bases, wherein the first segment and the second segment are separated by at least two DNA bases, and optionally, wherein the sequence of at least one of the chimeric primers further comprises a third segment comprising at least two adjacent RNA bases, wherein the second segment and the third segment are separated by at least two DNA bases,
wherein the reaction mixture comprises two chimeric primers, and each chimeric primer is an oligonucleotide comprising DNA and RNA bases with a sequence that includes a first segment comprising at least two adjacent RNA bases,
wherein the one or more chimeric primers in the reaction mixture comprise:
a forward primer configured to hybridize to an upstream portion of the template DNA molecule; and
a reverse primer configured to hybridize to a downstream portion of the template DNA molecule; wherein the forward primer and the reverse primer are chimeric oligonucleotides, each comprising DNA and RNA bases and a sequence that includes a first segment comprising at least two adjacent RNA bases, or
wherein the one or more chimeric primers are configured to amplify at least a portion of the template DNA while reducing or eliminating formation of non-specific amplification products,
wherein one or more chimeric primers are configured to amplify at least a portion of the template DNA while reducing or eliminating primer dimers and/or off-target amplification,
wherein the PCR assay is a multiplex PCR assay that generates an end product comprising:
a) less than 5% primer dimer amplification products;
b) less than 7% primer dimer amplification products; or
c) less than 10% primer dimer amplification products,
wherein at least one of the chimeric primers has a sequence consisting of a) DNA bases, and b) a pair of adjacent RNA bases at positions 3 and 4, as measured from the 3′ end of the chimeric primer,
wherein at least one of the chimeric primers has a sequence consisting of a) DNA bases, and b) a pair of adjacent RNA bases at positions 7 and 8, as measured from the 3′ end of the chimeric primer,
wherein at least one of the chimeric primers has a sequence consisting of a) DNA bases, and b) a pair of adjacent RNA bases at positions 14 and 15, as measured from the 3′ end of the chimeric primer,
wherein at least one of the RNA bases of the chimeric primer is replaced by an RNA analog, or
wherein at least one of the RNA bases of the chimeric primer is replaced by 2′-O-Methyl RNA.

22. (canceled)

23. (canceled)

24. (canceled)

25. The method of claim 21, wherein the first segment of each chimeric primer is located within 15 bases of the 3′ end of the sequence of each respective chimeric primer, each sequence having a DNA base at its 3′ end or wherein the sequence of each chimeric primer further comprises a second segment comprising at least two adjacent RNA bases, wherein the first segment and the second segment are separated by at least two DNA base, and optionally, wherein the sequence of each chimeric primer further comprises a third segment comprising at least two adjacent RNA bases, wherein the second segment and the third segment are separated by at least two DNA bases.

26. (canceled)

27. (canceled)

28. The method of claim 25, wherein at least one of the first segment, the second segment, and the third segment are located within 15 bases of the 3′ end of the sequence of each chimeric primer.

29. (canceled)

30. The method of claim 21, wherein the forward primer and the reverse primer each comprise a sequence having a DNA base at its 3′ end, and wherein the first segment of each sequence is located within 15 bases of its respective 3′ end.

31. The method of claim 21, wherein at least one of the forward primer and the reverse primer further comprises a second segment comprising at least two adjacent RNA bases, wherein the first segment and the second segment are separated by at least two DNA bases.

32. The method of claim 31, wherein both the forward primer and the reverse primer each further comprise a second segment comprising at least two adjacent RNA bases, wherein the first segment and the second segment are separated by at least two DNA bases.

33. (canceled)

34. (canceled)

35. (canceled)

36. (canceled)

37. (canceled)

38. (canceled)

39. (canceled)

40. (canceled)

41. A kit for performing the method claim 20, said kit comprising the one or more chimeric primers, and optionally one or more of: a) a DNA-dependent polymerase, b) a buffer compatible with a PCR assay, and c) deoxyribonucleotide triphosphates (dNTPs).
